(19) United States
(12) Patent Application Publication
(10) Pub. No.: US 2004/0208889 A1
(43) Pub. Date: Oct. 21, 2004

(54) Pharmaceutical use for secreted bacterial effector proteins

(76) Inventors: John Mark Sutton, Salisbury (GB); Clifford Charles Shone, Salisbury (GB)

Correspondence Address: Sterne Kessler, Goldstein & Fox, Suite 600, 1100 New York Avenue NW, Washington, DC, 20005-3934, US

(21) Appl. No.: 10/478,516

(22) PCT Filed: May 21, 2002
(86) PCT No.: PCT/GB02/02384

(51) Int. Cl. A61K 39/02; C07K 14/195

(52) U.S. Cl. 424/190.1; 530/350

(57) ABSTRACT

A polypeptide conjugate contains a bacterial injectable effector protein, secreted by a modified pilus or “needle-like” structure comprising a type m or type TV secretion apparatus, and a carrier that targets the conjugate to a target cell. The effector protein is used for a variety of purposes including treatment of neurodegenerative disease, intracellular infection and diseases associated with defects of secretion.

The present invention relates to pharmaceutical use of secreted, injected bacterial effector proteins. In particular, the present invention relates to manufacture and use of such proteins and combination and conjugation of the proteins with carriers.

A number of deficiencies exist in the availability and suitability of neuronal therapies. At the present time, a large number of neuronal disorders have inadequate provisions for therapeutic intervention. For example there is currently no effective treatment for neuronal damage caused by ischemia or trauma. Other neurodegenerative disorders such as Motor neurone disease, Alzheimer's disease, Parkinson's disease and prion disorders such as CJD are all poorly addressed by current therapies. This reflects in part the complexity of the nervous system and the difficulties in targeting suitable therapies to the specific cells affected. Neuronal repair after damage is another disorder for which there is no effective treatment.

A number of neurological disorders are known that arise from neuronal trauma that stimulates nerve damage due to internal processes such as apoptosis. It is known to treat such disorders using a superoxide dismutase in combination with a components that targets the enzyme to neurons. However, further active compounds for treatment of neuronal disease are desired.

It is known to use type III effectors in pharmaceutical compositions.

U.S. Pat. No. 5,972,899 describes a composition comprising Shigella IpaB, an IpaB fusion protein or a functional derivative or antagonist, or IpaB DNA for delivery to a eukaryotic cell to induce or to inhibit apoptosis. Site-specific delivery may be achieved within a targeted immunoliposome. Cell-type specificity is achieved by the incorporation of a cell-type selective monoclonal antibody into the lipid bilayer. Disadvantages associated with this delivery method include the very large size, low stability and poor tissue penetration of immunoliposomes, and difficulties associated with consistent immunoliposome manufacture for therapeutic use. There is also the likelihood of a high background effect due to fusion of immunoliposomes with non-target cell types, caused by the inherent properties of the liposome membrane.

WO 01/19393 describes Type III effector proteins linked to a protein transduction domain of the HIV TAT protein. DNA constructs encoding the effector-transducer fusion protein are targeted to host cells comprising a Type III secretion system using a tissue-specific viral or plasmid vector. Upon expression in the transformed host cells, the effector-transducer conjugate is secreted and undergoes secondary redistribution and uptake by neighbouring cells.

The HIV TAT transduction domain is not specific to any cell type, hence, targeting of effector is carried out solely at the DNA level. Disadvantages of targeting effector DNA (rather than targeting effector protein) include the time lag for processing of effector DNA to effector protein. Where viral vectors are used, there are the risks of immunogenic effects and of the vector integrating into the genome.

WO 00/37493 describes Bordetella pertussis effector virulence genes associated with a Type III secretion system. The pathogenicity genes or encoded polypeptides are used in vaccine compositions and may be conjugated to another molecule or provided with a carrier for delivery. Pathogenicity polypeptide may be delivered via a vector directing expression of Bordetella pathogenicity polynucleotide in vivo.

WO 98/56817 describes pharmaceutical compositions comprising a non-pathogenic organism expressing the YopJ protein, and YopJ protein combined with a carrier, for delivery of YopJ to gastrointestinal cells from the gut. The delivery mechanism disclosed in this document is via the normal bacterial Type III secretion system—that is, one step from bacterium to target cell.

WO 99/52563 describes targeting of proteins produced by recombinant Yersinia to the cytosol of eukaryotic cells for diagnostic/therapeutic purposes. Fusion proteins with the YopE targeting signal are expressed in Yersinia cells and delivered directly to eukaryotic cells via the Type III secretion system in the presence of the SycE chaperone.

U.S. Pat. No. 5,965,381 describes the in vitro use of recombinant Yersinia to deliver proteins to eukaryotic cells for immune diagnostic and therapeutic purposes. The proteins are fused to a delivery sequence, recognised by the Yersinia Type III secretion system.

It is not advantageous to make use of bacteria for delivering therapeutic proteins due to the risk of iliciting an unwanted immune response.

The present invention has as an object the provision of new pharmaceutical compositions for a variety of uses. A further object is to provide new pharmaceutical compositions for treatment of neuronal cells.

Accordingly, the present invention provides new therapies based upon a new class of bacterial-derived proteins, though the scope of the invention is intended to embrace also fragments and derivatives and modifications thereof that retain the properties of the native proteins.

A first aspect of the invention thus lies in a pharmaceutical composition, comprising a bacterial injected effector secreted by the type III or IV secretion pathway.

The pharmaceutical composition can be used for treatment of a subpopulation of cells in a patient, especially for a treatment selected from promoting survival of cells, preventing damage to cells, reversing damage to cells, promoting growth of cells, inhibiting apoptosis, inhibiting release of an inflammatory mediator from cells and promoting division of cells, or for a treatment selected from inhibiting survival of cells, inhibiting growth of cells, inhibiting division of cells, promoting apoptosis, killing cells, promoting release of an inflammatory mediator from cells and regulating nitric oxide release from cells.

A carrier can be provided to target the effector protein to a target cell, optionally targeting the effector to a cell selected from an epithelial cell, a neuronal cell, a secretory cell, an immunological cell, an endocrine cell, an inflammatory cell, an exocrine cell, a bone cell and a cell of the cardiovascular system.

Another means of delivery of the effector is via a conjugate of the effector protein and the carrier, the two suitably linked by a linker. One particularly preferred linker is cleavable, in that it can be cleaved after entry into the target cell so as to release the effector from the carrier. This linker can be a disulphide bridge or a peptide sequence including a site for a protease found in the target cell. In another embodiment of the invention, the linker is composed of two cooperating proteins, a first cooperating protein associated with the effector and the second associated with the cell targetting component. These respective parts can be administered separately and combine in vivo to link the effector to the cell targetting component. An example of such a two-part linker is botulinum toxin C2₁ in cooperation with C2₂.

In one embodiment of the invention, described in more detail below, a composition comprises a neuronal cell targeting component, linked by a cleavable linker to the effector protein. Preferably, the neuronal cell targeting component comprises a first domain targeting the effector to a neuronal cell and a second domain that translocates the effector into the cytosol of the neuronal cell.

Preparation of the compositions of the invention can be by combining a type III effector protein with a pharmaceutically acceptable carrier. In such compositions, the effector protein may be on its own or may be chemically linked with a (targetting) carrier. Another preparation method is to express a DNA that encodes a polypeptide having a first region that corresponds to the effector protein and a second region that codes for the carrier. A third region, between the first and second regions, which is cleaved by a proteolytic enzyme present in the target cell is optionally included.

A specific composition of the invention, for delivery of a bacterial type III effector protein to neuronal cells, comprises:

the effector protein; linked by a cleavable linker to

a neuronal cell targeting component, comprising a first domain that binds to a neuronal cell and a second domain that translocates the effector protein of the composition into the neuronal cell. It is preferred that the first domain is selected from (a) neuronal cell binding domains of clostridial toxins; and (b) fragments, variants and derivatives of the domains in (a) that substantially retain the neuronal cell binding activity of the domains of (a).

It is further preferred that the second domain is selected from (a) domains of clostridial neurotoxins that translocate polypeptide sequences into cells, and (b) fragments, variants and derivatives of the domains of (a) that substantially retain the translocating activity of the domains of (a).

In use of a composition of the invention for treatment of a neuronal condition, the linker is cleaved in the neuronal cell so as to release the effector protein from the targeting component, thus enabling the effector to have effect in the cell without being hindered by attachment to the targeting component.

Hence, also, the invention provides a method of delivering a bacterial type III effector protein to a neuronal cell comprising administering a composition of the invention.

The first domain may suitably be selected from (a) neuronal cell binding domains of clostridial toxins; and (b) fragments, variants and derivatives of the domains in (a) that substantially retain the neuronal cell binding activity of the domains of (a). The second domain is suitably selected from (a) domains of clostridial neurotoxins that translocate polypeptide sequences into cells, and (b) fragments, variants and derivatives of the domains of (a) that substantially retain the translocating activity of the domains of (a). The second domain is further suitably selected from:

(a) a translocation domain that is not a H_{N} domain of a clostridial toxin and is not a fragment or derivative of a H_{N} domain of a clostridial toxin;

(b) a non-aggregating translocation domain as measured by size in physiological buffers;

(c) a H_{N} domain of a diphtheria toxin,

(d) a fragment or derivative of (c) that substantially retains the translocating activity of the H_{N} domain of a diphtheria toxin,

(e) a fusogenic peptide,

(f) a membrane disrupting peptide, and

(g) translocating fragments and derivatives of (e) and (f).

In an embodiment of the invention a construct comprises effector protein linked by a disulphide bridge to a neuronal cell targeting component comprising a first domain that binds to a neuronal cell and a second domain that translocates the effector protein into the neuronal cell. This construct is made recombinantly as a single polypeptide having a cysteine residue on the effector protein which forms a disulphide bridge with a cysteine residue on the second domain. The effector protein is covalently linked, initially, to the second domain. Following expression of this single polypeptide, effector protein is cleaved from the second domain leaving the effector protein linked only by the disulphide bridge to the rest of the construct.

Particular aspects of the invention reside in further choices for the binding and translocation domains, and one such aspect provides a non-toxic polypeptide, for delivery of the effector protein to a neuronal cell, comprising:

a binding domain that binds to the neuronal cell, and

a translocation domain that translocates the effector protein into the neuronal cell,

wherein the translocation domain is not a H_{N} domain of a clostridial neurotoxin and is not a fragment or derivative of a H_{N} domain of a clostridial toxin.

The binding domain is suitably comprised of or derived from clostridial heavy chain fragments or modified clostridial heavy chain fragments. As used herein, the term “modified clostridial heavy chain fragment” means a polypeptide fragment that retains similar biological functions to the corresponding heavy chain of a botulinum or tetanus neurotoxin but differs in its amino acid sequence and other properties compared to the corresponding heavy chain. The invention more specifically provides such constructs that are based on fragments derived from botulinum and tetanus neurotoxins.

In a further aspect, the invention also provides a polypeptide, for delivery of a effector protein to a neuronal cell, comprising:

a binding domain that binds to the neuronal cell, and

a translocation domain that translocates the effector protein into the neuronal cell,

wherein the resulting construct is non-aggregating.

Whether the construct is an aggregating one is usually apparent from a lack of solubility of the construct, and this may be seen upon simple visual inspection of the construct in aqueous media: non-aggregating domains result in constructs of the invention that are partially or preferably totally soluble whereas aggregating domains result in non-soluble aggregates of polypeptides having apparent sizes of many tens or even hundreds the size of a single polypeptide. Generally, the construct should be non-aggregating as measured by its size on gel electrophoresis, and domain sizes or apparent domain sizes thus measured should preferably be less than 1.0×10⁶ daltons, more preferably less than 3.0×10⁵ daltons, with the measuring being suitably carried out on native PAGE using physiological conditions.

A still further aspect of the invention provides a polypeptide, for delivery of a effector protein to a neuronal cell, comprising:

a binding domain that binds to the neuronal cell, and

a translocation domain that translocates the effector protein into the neuronal cell,

wherein the translocation domain is selected from (1) a H_{N} domain of a diphtheria toxin, (2) a fragment or derivative of (1) that substantially retains the translocating activity of the H_{N} domain of a diphtheria toxin, (3) a fusogenic peptide, (4) a membrane disrupting peptide, (5) a H_{N} from botulinum toxin C₂ and (6) translocating fragments and derivatives of (3), (4) and (5).

It is to be noted that botulinum toxin C₂ is not a neurotoxin as it has no neuronal specificity, instead it is an enterotoxin and suitable for use in the invention to provide a non-aggregating translocation domain.

A yet further aspect of the invention provides a polypeptide, for delivery of a effector protein to a neuronal cell, comprising:

a binding domain that binds to the neuronal cell, and

a translocation domain that translocates the effector protein into the neuronal cell,

wherein the polypeptide has reduced affinity to neutralising antibodies to tetanus toxin compared with the affinity to such antibodies of native tetanus toxin heavy chain.

The above aspects may singly or in any combination be exhibited by polypeptides of the invention and thus a typical preferred polypeptide of the invention (i) lacks the neurotoxic activities of botulinum and tetanus toxins, (ii) displays high affinity to neuronal cells corresponding to the affinity of a clostridial neurotoxin for those cells, (iii) contains a domain which can effect translocation across cell membranes, and (iv) occurs in a less aggregated state than the corresponding heavy chain from botulinum or tetanus toxin in physiological buffers.

A significant advantage of the polypeptides of particular aspects of the invention is their non-aggregated state, thus rendering them more usable as soluble polypeptides. The polypeptides according to the invention generally include sequences from the H_{C} domains of the botulinum and tetanus neurotoxins and these are combined with functional domains from other proteins, such that the essential functions of the native heavy chains are retained. Thus, for example, the H_{C} domain of botulinum type F neurotoxin is fused to the translocation domain derived from diphtheria toxin to give modified clostridial heavy chain fragment. Surprisingly, such polypeptides are more useful as constructs for delivering substances to neuronal cells than are the native clostridial heavy chains.

The current invention provides constructs containing type III secreted effector proteins and optionally other functional domains that effect the specific delivery of the type III effector moiety to neuronal cells These constructs have a variety of clinical uses for the treatment of neuronal diseases.

The type III secretion mechanism of Gram negative bacterial pathogens is a complex system used to deliver proteins to eukaryotic cells. The secretion mechanism utilises at least 10-15 essential proteins to form an injection needle that extends from the surface of the bacteria and penetrates into the host cell. The effector proteins are then trafficked across the bacterial and host membranes through the lumen of the needle and injected directly into the cell cytosol. This process involves a still undefined secretion signal and involves specific chaperone proteins that deliver the effector to the secretion machinery. The system delivers a wide range of protein effectors capable of modulating host cell function in such a way as to allow the persistence or spread of the pathogen in the host. These effectors modulate a number of signalling pathways and one pathogen may export several effectors that regulate different pathways either concurrently or during different phases of its life cycle. Type III secretion systems have been described in a wide range of pathogenic bacteria including but not restricted to:

Mammalian pathogens; Yersinia species (including pestis, pseudotuberculosis, enterocolitica), Salmonella species (including typhimurium, enterica, dublin, typhi) Escherichia coli, Shigella species (e.g. flexnen), Pseudomonas aeruginosa, Chiamydia species (e.g. pneumoniae, trachomatis), and Bordetella species, and Burkholderia species

Plant pathogens; Pseudomonas syringae, Erwinia species, Xanthomonas species, Ralstonia solanacearum, and Rhizobium species

Insect pathogens; Sodalis glossinidius, Edwardsiella ictaluri, and Plesiomonas species

Effector proteins from any of these species, whether mammalian pathogens or not, have therapeutic potential for treating human or animal disease.

Table 1 lists a number of type III effectors that have been identified to date.

The type IV secretion system shows a remarkable degree of similarity to the type III system in that it forms a needle-like structure through which effector proteins are injected into the host cell cytoplasm. However, the proteins involved in the structure of the needle are different for the two systems and the effectors are also divergent. The effectors function to modulate cellular signalling to establish and maintain the intracellular niche and/or promote invasion and proliferation. The system is described as essential in a number of important bacterial pathogens including Legionella pneumophila, Bordetella pertussis, Actinobacillus actinomycetemcomitans, Bartonella henselae, Escherichia coli, Helicobacter pylori, Coxiella burnetii, Brucella abortus, Neisseria species and Rickettsia species (e.g. prowazekii). Similar type IV secretion systems exist in plant or invertebrate pathogens and are also a source of therapeutic agents. A number of described type IV effectors are also listed in table 1 with proposed functions.

The function of a variety of type III effectors has been described in recent years. Interestingly a number of effectors from different organisms have evolved to target particular signalling pathways suggesting some similarities in the mechanism of pathogenicity. The precise specificity of particular effectors may vary according to pathogen and cell type and this range of activities make them attractive candidates for therapeutic applications. Examples of some of the families of effectors useful in the present invention are described below:

GTPase activating proteins. YopE from Yersinia pseudotuberculosis, SptP from Salmonella typhimurium and ExoS and ExoT from Pseudomonas aeruginosa are all GTPase activating proteins (GAPs) for Rho family GTPases and are characterised by a conserved “arginine finger” domain (Black and Bliska, (2000) Molecular Microbiology 37:515-527; Fu and Galan (1999) Nature 401:293-297; Goehring et al (1999) Journal of Biological Chemistry 274:36369-36372). By increasing the hydrolysis of bound GTP they promote the formation of the inactive GDP-bound of the GTPase. This acts to down-regulate the function of a range of GTPases in cells. YopE is a 23 kDa effector which is translocated into the cytosol of cells during infection by Y.pseudotuberculosis and other strains. Studies in vitro have shown that it acts as a GAP for RhoA, Cdc42 and Rac1, but not for Ras (Black and Bliska, (2000) Molecular Microbiology 37:515-527). A point mutation within the arginine finger motif causes a loss of GAP activity and this correlates directly with its biological activity in cells. In in vivo studies carried out using a cell model that mimics the normal site of Yersinia infection YopE appears to have a greater specificity for Cdc42 (Andor et al (2001) Cellular Microbiology 3:301-310). The GAP activity of SptP shows greater specificity for Cdc42 and Rac1 compared to RhoA. The GAP activity of particular proteins is likely to vary for different cell types and delivery routes. SptP, ExoS and ExoT are bifunctional enzymes with additional enzymatic domains (SptP, tyrosine phosphatase; ExoS, ExoT, ADP-ribosyltransferase). In the case of ExoS this activity blocks the activation of Ras GTPase allowing a co-ordinated modulation of different signalling pathways (Henriksson et al (2000) Biochemical Journal 347:217-222).

Guanine nucleotide exchanae factor. SopE and SopE2 from Salmonella typhimurium and related proteins act as guanine nucleotide exchange factors (GEFs) for a range of GTPases (Hardt et al (1998) Cell 93:815). GEFs function by enhancing the rate of replacement of bound GDP by GTP causing the activation of the GTPase. This effectively upregulates the activity of specific GTPases in the cell. Native SopE is a 240 amino acid protein which is injected into the host cell cytosol by S.typhimurium. The N-terminal 77 amino acids of the protein act as a secretion signal and are dispensable for the biological activity of the protein (Hardt et al (1998) Cell 93:815). In in vitro studies SopE acts as a GEF for CDc42, Rac1, Rac2, RhoA, and RhoG. Cellular GEFs show a high degree of specificity for particular GTPases and it is likely that SopE will show greater specificity in vivo. This specificity is likely to vary according to cell type and delivery route. The type IV effector, RalF, from Legionella pneumophila is a further exchange factor affecting the function of small GTPases. In this case the target is the ADP ribosylation factor (ARF) family and this is the first example of a bacterial effector that targets this family (Nagai et al (2002) Science 295;679-682).

Covalent modification of GTPase. The type III effector YopT from Y.pestis and certain other Yersinia strains has similar effects in vivo to YopE (Iriarte and Cornelis (1998) Molecular Microbiology 29:915-929). In HeLa cells YopT causes a shift in the electrophoretic mobility of RhoA but not Cdc-42 or Rac (Zumbihl et al (1999) Journal of Biological Chemistry 274:29289-29293). It is still not known whether this represents a direct modification of RhoA by YopT or whether other cellular factors are involved. The specificity of YopT for RhoA offers significant therapeutic possibilities.

Regulation of cell signalling via protein kinase and phosghatase. YopO/YpkA from Yersinia spp are protein kinase related to eukaryotic serine/threonine kinases (Galyov et al (1993) Nature 361:730-732). YopO/YpkA causes a similar cell rounding to that observed for other effectors such as YopE suggesting a role in modulating GTPase function. The small GTPases RhoA and RacI have been shown to bind to YopO and YpkA suggesting that these are the intracellular targets for the kinase (Barz C et al (2000) FEBS Letters 482:139-143). The type IV effector CagA from Helicobacter pylori also affects the cytoskeleton of infected cells and its activity is dependent on its phosphorylation by intracellular kinases. CagA functions via the SHP-2 tyrosine phosphatase to modulate downstream signalling.

Inositol phosphatases. SigD from Salmonella typhimurium, SopB from S.dublin and IpgD from Shigella flexneri are all putative inositol phosphatases. In intestinal cells SopB causes an accumulation of inositol 1,4,5,6, tetrakisphosphate. Mutations in active site of SopB abolishes both its phopshatase activity and the accumulation of inositol tetrakisphosphate (Norris et al (1998) Proceedings of the National Academy of Science U.S.A 95:14057-14059). SopB appears to hydrolyse a wide range of inositol and phosphatidylinositol phosphates in vitro although its precise intracellular target remains to be defined (Eckmann et al (1997) Proceedings of the National Academy of Science U.S.A 94:14456-14460). SigD appears to have a different specificity in vivo as it does not lead to an increase in the levels of inositol 1,4,5,6, tetrakisphosphate (Eckmann et al (1997)). Although again the precise intracellular target has not been defined, SigD has been shown to lead to the activation of Akt/Protein kinase B in epithelial cells (Steele-Mortimer (2000) Journal of Biological Chemistry 275:37718-37724). The activity has been shown to be dependent on the presence of a synaptojanin-homologous region close to the C-terminus of the protein (Marcus et al (2001) FEBS letters 494:201-207). The homologous protein IpgD also stimulates the activation of Akt in these cells (Marcus et al (2001)). The potential to activate Akt offers a number of therapeutic opportunities as it is a key regulator of cellular survival (reviewed in Vanhaesebroeck and Alessi (2000) Biochemical Journal 346:561-576).

Inhibition of mitogen-activated protein kinase kinase. YopJ from Yersinia pestis is another translocated effector with a wide range of homologs including AvrA from Salmonella spp. and a variety of effectors from plant pathogens. YopJ has been shown to inactivate a broad range of mitogen-activated protein kinase kinases (MKKs) (Orth et al (1999) Science 285:1920-1923) causing apoptosis in macrophages. YopJ is suggested to act as a ubiquitin-like protein protease causing increased turnover of signalling molecules via removal of a Sumo-1 tag from the MKK (Orth et al (2000) Science 290:1594-1597). Interestingly in cell models of cytokine production and macrophage killing AvrA shows no activity despite its homology to YopJ suggesting that the specificity of the proteins may be different (Schesser K et al (2000) Microbial Pathogenesis 28:59-70). In neuronal cells these different specificities may offer potential therapeutic applications for modulating MKKs involved in apoptosis or inflammatory responses.

Modulators of cellular trafficking. SpiC from Salmonella enterica inhibits the fusion of endosomal vesicles to prevent the exposure of Salmonella to lyosomal degradation (Uchiya et al (1999) EMBO Journal 18:3924-3933). The ability to modulate intracellular trafficking pathways offers a number of therapeutic opportunities for modulating cycling of receptors or release of material from membrane bound vesicles.

A number of additional effector proteins are implicated in regulating and maintaining the intracellular compartments occupied by bacterial pathogens. Salmonella, in common with many other pathogens, establishes a specialised intracellular compartments. Salmonella has a dedicated type III secretion system that secretes proteins into the host cell cytosol from within this compartment and the effectors secreted by this system (including SpiC, SopE/E2, SseE,F,G,J, PipA,B, SifA,B)maintain the integrity of this compartment. A recent paper described the synergistic effects of SseJ and SifA in regulating processes from the vacuolar membrane (Ruiz-Albert et al (2002) Molecular microbiology 44;p645-661). These proteins and their counterparts from other intracellular pathogens have significant potential for treating disorders affecting intracellular trafficking pathways. RalF and a number of the other effectors described previously may also have significant therapeutic potential for such disorders.

The botulinum neurotoxins are a family of seven structurally similar, yet antigenically different, protein toxins whose primary site of action is the neuromuscular junction where they block the release of the transmitter acetylcholine. The action of these toxins on the peripheral nervous system of man and animals results in the syndrome botulism, which is characterised by widespread flaccid muscular paralysis (Shone (1986) in ‘Natural Toxicants in Foods’, Editor D. Watson, Ellis Harwood, UK). Each of the botulinum neurotoxins consist of two disulphide-linked subunits; a 100 kDa heavy subunit which plays a role in the initial binding and internalisation of the neurotoxin into the nerve ending (Dolly et. al. (1984) Nature, 307, 457460) and a 50 kDa light subunit which acts intracellularly to block the exocytosis process (McInnes and Dolly (1990) Febs Lett., 261, 323-326; de Paiva and Dolly (1990) Febs Lett., 277, 171-174). Thus it is the heavy chains of the botulinum neurotoxins that impart their remarkable neuronal specificity.

Tetanus toxin is structurally very similar to botulinum neurotoxins but its primary site of action is the central nervous system where it blocks the release of inhibitory neurotransmitters from central synapses (Renshaw cells). As described for the botulinum toxins above, it is domains within the heavy chain of tetanus toxin that bind to receptors on neuronal cells.

The binding and internalisation (translocation) functions of the clostridial neurotoxin (tetanus and botulinum) heavy chains can be assigned to at least two domains within their structures. The initial binding step is energy-independent and appears to be mediated by one or more domains within the H_{C} fragment of the neurotoxin (C-terminal fragment of approximately 50 kDa) (Shone et al. (1985), Eur. J. Biochem., 151,75-82) while the translocation step is energy-dependent and appears to be mediated by one or more domains within the H_{N} fragment of the neurotoxin (N-terminal fragment of approximately 50 kDa).

Isolated heavy chains are non-toxic compared to the native neurotoxins and yet retain the high affinity binding for neuronal cells. Tetanus and the botulinum neurotoxins from most of the seven serotypes, together with their derived heavy chains, have been shown to bind a wide variety of neuronal cell types with high affinities in the nM range (e.g. botulinum type B neurotoxin; Evans et al. (1986) Eur. J. Biochem. 154, 409-416). Another key characteristic of the binding of the tetanus and botulinum heavy chains to neuronal cells is that the receptor ligand recognised by the various toxin serotypes differ. Thus for example, botulinum type A heavy chain binds to a different receptor to botulinum type F heavy chain and these two ligands are non-competitive with respect to their binding to neuronal cells (Wadsworth et al. (1990), Biochem J. 268, 123-128). Of the clostridial neurotoxin serotypes so far characterised (tetanus, botulinum A, B, C₁, D, E and F), all appear to recognise distinct receptor populations on neuronal cells. Collectively, the clostridial neurotoxin heavy chains provide high affinity binding ligands that recognise a whole family of receptors that are specific to neuronal cells.

The present invention also provides constructs for the delivery of type III effector proteins specifically to neuronal cells. The mechanism by which the type III effector protein is delivered to the cell by these constructs is completely different to that used by the host bacteria. Instead of being injected directly into the cellular cytosol, specific constructs of the invention deliver the type III effector protein to cells via a number of sequentially acting biologically active domains and by a process resembling receptor-mediated endocytosis. Surprisingly, when delivered by this completely different mechanism, the type III effector proteins are biologically active within the cellular cytosol.

Particular constructs of the invention comprise three functional domains defined by their biological activities. These are:

the type III effector moiety (for examples see Table1);

a targeting domain that binds the construct to receptors and that provides a high degree of specificity to neuronal cells; and

a translocation domain that after internalisation of the construct, effects the translocation of the type III effector moiety through the endosomal membrane into the cell cytosol.

The type III effector-containing construct may also contain ‘linker proteins’ by which these domains are interconnected. In one embodiment of the invention the type III effector moiety is linked to the translocation domain via a disulphide bridge.

In a preferred embodiment of the invention, the targeting domain is derived from a clostridial neurotoxin binding fragment (H_{c} domain). This may be derived from tetanus toxin or any one of the eight botulinum toxin serotypes (A-G). Delivery via the clostridial neurotoxin receptors differs significantly to the normal delivery route of the type III effectors and offers a number of advantages:

The clostridial H_{c} fragments bind with high affinity to receptors on the cell surface and provide high specificity to neuronal cells. The clostridial neurotoxins are internalised via an acidic endosome which triggers the translocation of the type III effector moiety across the membrane and into the cytosol.

For non-neuronal cells a wide range of high affinity binding domains have been defined for specific cell types. Examples are described for a number of cellular targets.

The agent can comprise a ligand or targeting domain, which binds to an endocrine cell and is thus rendered specific for these cell types. Examples of suitable ligands include iodine; thyroid stimulating hormone (TSH); TSH receptor antibodies; antibodies to the islet-specific monosialo-ganglioside GM2-1; insulin, insulin-like growth factor and antibodies to the receptors of both; TSH releasing hormone (protirelin) and antibodies to its receptor; FSH/LH releasing hormone (gonadorelin) and antibodies to its receptor; corticotrophin releasing hormone (CRH) and antibodies to its receptor; and ACTH and antibodies to its receptor.

Ligands suitable to target an agent to inflammatory cells include (i) for mast cells, complement receptors in general, including C4 domain of the Fc IgE, and antibodies/ligands to the C3a/C4a-R complement receptor; (ii) for eosinophils, antibodies/ligands to the C3a/C4a-R complement receptor, anti VLA-4 monoclonal antibody, anti-IL5 receptor, antigens or antibodies reactive toward CR4 complement receptor; (iii) for macrophages and monocytes, macrophage stimulating factor, (iv) for macrophages, monocytes and neutrophils, bacterial LPS and yeast B-glucans which bind to CR3, (v) for neutrophils, antibody to OX42, an antigen associated with the iC3b complement receptor, or IL8; (vi) for fibroblasts, mannose 6-phosphate/insulin-like growth factor-beta (M6P/IGF-II) receptor and PA2.26, antibody to a cell-surface receptor for active fibroblasts in mice.

Ligands suitable to target an agent to exocrine cells include pituitary adenyl cyclase activating peptide (PACAP-38) or an antibody to its receptor.

Ligands suitable to target an agent to immunological cells include Epstein Barr virus fragment/surface feature or idiotypic antibody (binds to CR2 receptor on B-lymphocytes and lymph node follicular dendritic cells).

Suitable ligands for targeting platelets for the treatment of disease states involving inappropriate platelet activation and thrombus formation include thrombin and TRAP (thrombin receptor agonist peptide) or antibodies to CD31/PECAM-1, CD24 or CD106NCAM-1, and ligands for targeting cardiovascular endothelial cells for the treatment of hypertension include GP1b surface antigen recognising antibodies.

Suitable ligands for targeting osteoblasts for the treatment of a disease selected from osteopetrosis and osteoporosis include calcitonin, and for targeting an agent to osteoclasts include osteoclast differentiation factors (eg. TRANCE, or RANKL or OPGL), and an antibody to the receptor RANK.

In one embodiment of the invention the translocation domain is derived from a bacterial toxin. Examples of suitable translocation domains are those derived from the clostridial neurotoxins or diphtheria toxin.

In another embodiment of the invention, the translocation domain is a membrane disrupting or ‘fusogenic’ peptide, which functions as a translocation domain. An example of such a peptide is that derived from influenza virus haemagglutinin HA2 (residues 1-23).

In one example of the construct of the invention, the type III effector protein is SigD from Salmonella spp. In another example of the construct of the invention, the type III effector protein is YopE from Yersinia spp.

In an example of the construct of the invention in which the type III effector moiety is SigD from Salmonella spp, the construct may consist of the following:

the SigD type III effector moiety;

the translocation domain from diphtheria toxin;

the binding domain (H_{c} domain) from botulinum type A neurotoxin; and

a linker peptide to enable attachment of the SigD effector to the translocation domain via a disulphide bridge.

In an another example of the construct of the invention in which the type III effector moiety is SigD from Salmonella spp, the construct consists of the following:

the SigD type III effector moiety;

the translocation domain in the form of a fusogenic peptide;

the binding domain (H_{c} domain) from botulinum type F neurotoxin; and

a linker peptide to enable attachment of the SigD effector to the translocation domain via a disulphide bridge.

In an example of the construct of the invention in which the type III effector moiety is YopE from Yersinia spp, the construct may consist of the following:

the YopE type III effector moiety;

the translocation domain from diphtheria toxin;

the binding domain (H_{c} domain) from botulinum type F neurotoxin; and

a linker peptide to enable attachment of the YopE effector to the translocation domain via a disulphide bridge.

The invention enables manipulation of cell signalling, and in a specific example SigD is incorporated into a construct of the invention and can be used to promote neuronal cell survival under stress. By targeting the appropriate intracellular signalling pathway, it is possible to simultaneously regulate a number of pathways to improve the prospects for neuronal survival. SigD (also known as SopB) activates the protein kinase Akt, which is a key intermediate in the pro-survival signalling pathways mediated by various growth factors. Not only does Akt up-regulate pro-survival transcription factors such as NF-kB, but it also down-regulates several pro-apoptotic factors such as Bad and Forkhead.

A number of type III and IV effectors function to maintain the intracellular niche of the bacteria within the host cell. Whilst some bacterial pathogens are released into the cell cytosol, many form and maintain a specialised intracellular compartment sometimes termed a vacuole. One of the principle functions of many effector protein is to regulate the fusion of the compartment with other intracellular compartments such as potentially damaging phagolysosomal. At the same time the pathogen may need to promote fusion with other membrane bound compartments, including recycling endosomes, to either provide nutrients to the encapsulated pathogen or allow the dissemination of the pathogen to other locations. Intracellular pathogens offer a wide range of effector molecules for regulating intracellular trafficking and membrane fusion.

The mechanism underlying the fusion of membrane bound vesicles is conserved in a number of cellular processes. Broadly speaking, membrane fusion events are classified either as secretory processes for the release of material from the plasma membrane, or as endocytic processes that move material from the plasma membrane to the lysosomal system. This simplified classification does not take into account retrograde and anterograde processes, which occur within these pathways, or multiple points of communication between the two pathways. The underlying mechanism in all membrane fusion events can be broken down into 4 component phases:

The transported material is concentrated at a specific site on the donor membrane and is “pinched off” in a vesicle that becomes separated from this membrane.

The vesicle is transported to the acceptor membrane along cytoskeletal fibres (e.g. microtubules).

The vesicle then attaches to the acceptor membrane via a “docking/tethering” mechanism mediated by SNARE complex proteins.

The vesicle and the acceptor membrane fuse to release the contents of the vesicle through the acceptor membrane.

Thus similar SNARE proteins and regulatory proteins underpin the fusion of endosomal vesicles with the lysosome, endoplasmic reticulum with the Golgi and trans-Golgi network, and secretory vesicles with the plasma membrane. The functional conservation of the membrane fusion mechanism means that a bacterial effector protein that would normally regulate the fusion of a specific event can be directed to modulate other fusion events. For example, an effector that blocks endosomal fusion with the lysosome can be redirected to block the fusion of secretory vesicles with the plasma membrane, or ER vesicles with the Golgi network.

One of the key classes of regulatory proteins that have been defined in vesicle trafficking are small GTPases of the Ras superfamily termed Rab proteins (or Ypt proteins in yeast). Rab proteins are implicated in every stage of membrane fusion. For example Rab 1,2,5 and 9 are involved in sorting material for transport (stage 1 above), Rab5,6,27 and Sec4 mediate transport (stage 2), Rab1,5, Ypt1,7 Sec4 influence docking to the acceptor membrane (stage 3) and other Rab proteins implicated in promoting membrane fusion. The list above shows that certain Rab proteins, such as Rab1 and Rab5, are involved in more than 1 stage of the fusion process. Similarly some Rab proteins are present on all membrane vesicles whilst others have more specialised roles in specific fusion events.

Rab proteins are key potential targets for modification by either bacterial pathogens intent on blocking or promoting membrane fusion events or by therapeutic agents designed to regulate intracellular trafficking. One of the first effector proteins to be described as having an effect on Rab function was the secreted effector protein SopE2 from Salmonella species. SopE2 acts as a guanine nucleotide exchange factor for Rab5a resulting in increased activation of the protein on the cell membrane. This activity has been correlated with increased survival of Salmonella in infected HeLa cells and macrophages (Cell Micobiol. 3 p473). SpiC is another Salmonella effector that blocks endosome fusion (EMBO J. 18p3924-3933). Unlike SopE, which shows some conservation with normal cellular regulators of GTPase, SpiC shows no clear homology to other proteins. Its ability to block one of the four stages of vesicle fusion is known. It could exert its activity at the level of the SNARE proteins, modulate Rab function directly or operate at the level of one of the regulators of Rab function. Membrane insertion is essential for Rab activity. Rab proteins form a stable complex with Rab escort protein (REP) in the cytosol and this is a substrate for a geranyl geranyl transferase (RabGGT) which adds a C-terminal isoprenoid moiety. In the absence of REP or RabGGT the Rab protein would remain in an inactive form in the cytosol. REP also mediates the membrane insertion of the modified Rab into the donor membrane. Rab proteins can also be retrieved from the membrane via the action of Rab GDP dissociation inhibitor (RabGDI). All of these proteins are potential targets for bacterial pathogens to alter membrane fusion events. The precise effect would depend on whether alterations cause an increase or decrease in the levels of active Rab in the donor membrane, and the specificity for particular Rab proteins.

A number of human diseases have now been identified in which mutations affect either Rab proteins or their regulators. These human diseases serve to illustrate the cellular effects of alterations in Rab control in cells. Thus mutations in Rab27 (Griscelli syndrome), REP1 (choroiderma), RabGD1α (X-linked mental retardation) and RabGGT α subunit (Hermansky-Pudlack syndrome) are all implicated in human disease (as reviewed in Seabra et al Trends in Molecular Medicine (2002) 8;23-26, Olkkonen and Ikonen New England Journal of Medicine (2000) 343;1104)). A wide range of human diseases involve defects in intracellular trafficking (as reviewed in Aridor and Hannan Traffic (2000) 1;836-851). Modulation of membrane fusion via the specialised properties of bacterial effector proteins directed at one of the 4 mechanisms described above offers therapeutic opportunities for these diseases and others where transport properties are affected.

The targeting of the membrane fusion event between secretory vesicles and the plasma membrane allow the control of secretion from cells. Effectors that alter regulation of specific Rab proteins, either directly or via one of the mechanisms described above, including Rab3a,b,c and d, Rab8a and b, Rab26, Rab27a Rab37, or affect any of the other molecular events of membrane fusion (1-4 described above) can regulate secretion. Effector proteins can be applied to either increase or decrease secretion from a specific cell type. In a therapeutic context this is valuable for the treatment of a wide range of disorders including muscle spasms (blephorospasm, torticolis etc) hypersecretion disorders (COPD, bronchitis, asthma).

By modulating the fusion of recycling endosomes with either the lysosome or the plasma membrane it also possible to modulate the presentation of specific families of cell surface marker. Again effectors directed to alter regulation of specific Rab proteins, such as Rab4a and b, Rab11a and b, Rab15, Rab17, Rab18 or affect other molecular events in the fusion mechanism, can either up or down regulate presentation of cell surface marker. Therapeutically this has enormous potential for altering the response of cells to external stimuli (e.g. modulating response to growth factors, hormones, cytokines, chemokines or other signalling molecules), modifying the recognition of cells by external factors (e.g. immune surveillance) or for switching cell signalling pathways on or off.

Using constructs of the invention, therapeutic intervention can be provided in neurodegenerative disorders such as Alzheimer's disease and Prion diseases (vCJD). Both diseases are characterised by the accumulation of insoluble protein plaques due to misfolding of cellular proteins. In both cases an intracellular amplification of misfolded protein, via passage through endosomal-lysosomal compartments, is implicated in the progression of the disease. Neuronally targeted bacterial effectors as described herein, which modulate the fusion of endosomal and lysosomal compartments, allow control of the accumulation of insoluble protein. As this is one of the key survival strategies of many intracelullar bacterial pathogens, a number of therapeutic molecules are available, for example Salmonella effectors such as SpiC, SptP and SopE2.

In still further embodiments of the invention, constructs are provided for inhibition or promotion of secretion, containing a type III effector and a targetting moiety. Specific effectors for this purpose are selected from SpiC, SopE, RalF, Sse E, F, G and J, PipA, PipB, SifA and SifB. These constructs target the membrane fusion event between secretory vesicles and the plasma membrane to allow the control of secretion from cells. Effectors that alter regulation of specific Rab proteins, either directly or via one of the mechanisms described above, including Rab3a,b,c and d, Rab8a and b, Rab26, Rab27a Rab37, or affect any of the other molecular events of membrane fusion, can regulate secretion. Effector proteins can be applied to either increase or decrease secretion from a specific cell type. In a therapeutic context this is valuable for the treatment of a wide range of disorders including muscle spasms (blephorospasm, torticolis etc) hypersecretion disorders (COPD, bronchitis, asthma).

The pathogenic strategy to establish a specialised intracellular niche and to modulate fusion of that compartment with other vesicles is conserved for a vast range of pathogens. Not only does this provide a vast range of molecules capable of modulating the cellular events as described above, but it also provides an array of potential therapeutic targets for such molecules. Although many of the intracellular pathogens described in table 2 establish membrane bound compartments, the precise biochemistry and the signalling events and effectors needed to maintain these compartments are very different. A few intracellular pathogens escape from the phagosomal or endosomal compartment in which they enter the cell. The effector proteins involved in this process are incompatible with the life cycle of pathogens that remain in membrane compartments. The effector proteins of two intracellular pathogens existing in membrane bound vesicles are also not necessarily compatible. For example, enhancement of Rab5a activity by Salmonella in macrophages is correlated with enhanced survival (Cell Microbiology 3;473-). However, increases in Rab5a expression/activity accelerates intracellular destruction of Listeria monocytogenes in macrophages (J. Biological Chemistry 274;11459). The Salmonella effector proteins that are likely to be involved in Rab5a recruitment (e.g. SopE2, SpiC or other SPI-2 secreted effectors) are therefore potential therapeutic agents for treating intracellular Listeria.

In its crudest form anti-microbial therapy could involve treating one intracellular pathogen with a second pathogen on the basis that the two intracellular compartments and requirements of the organisms would not be compatible. For example treatment of TB infected macrophages with Salmonella might be expected to result in provoked “vacuole” lysosome fusion within the macrophage leading to the eradication of the TB. The type and fate of the super-infecting pathogen would have to be carefully chosen so as not to exacerbate the infectivity or spread of the original organism.

A refinement of the superinfection strategy would therefore focus on the targeted delivery of effector molecules to specific target cells as described by this invention. This could either utilise a highly attenuated pathogen (e.g. Salmonella that only secretes SopE2 or SptP) or targeted protein delivery (e.g. using a toxin delivery domain, antibody or similar cell targeting ligands). Protective antigen from Bacillus anthracis would be capable of targeting effectors to macrophages for the treatment of a wide range of bacterial pathogens. The specific addition of carbohydrate moieties will enable specific targeting of pools of macrophages via the mannose receptor (e.g Vyas et al, International Journal of Pharmaceutics (2000) 210p1-14). A cell surface marker specific for infected cells (as distinct from uninfected cells) would offer an ideal target for delivery systems. The cell type infected by the pathogen would determine the choice of delivery ligand whilst the precise fate of the cell compartment would determine the choice of effector (e.g. cell apoptosis, lysis, endosome-lysosome fusion, endosome acidification etc).

A key benefit of this type of therapy is that the effector proteins are not intrinsically toxic to the cell and therefore delivery of the protein to uninfected target cells is unlikely to have any deleterious effects. In this case, whilst desirable, the precise specificity of the targeting ligand is not essential for successful therapy.

The wide range of intracellular pathogens and the difficulty in treating/immunising against these organisms make this approach a valuable alternative to antibiotic therapy. The method is also attractive as avoidance of the antimicrobial agent either means that the pathogen must produce a molecule capable of overriding the effector-induced cell stimulus or must significantly modify its lifestyle. For obligate intracellular pathogens or where the intracellular stage is essential for propagation, this may offer greater hopes for extended antimicrobial use than current antibiotic strategies targeted at specific biochemical interactions.

In another example of the invention in which the effector protein is SpiC from Salmonella spp, the construct may consist of the following:

the SpiC effector moiety fused to a domain capable of interacting with protective antigen;

the protective antigen from Bacillus anthracis;

where the construct is either co-administered or where the SpiC moiety is administered after the protective antigen.

The constructs of the invention are preferably produced either wholly or partially by recombinant technology. In an embodiment of the invention where a construct of the invention is produced by recombinant technology, the construct of the invention will be produced as a single multi-domain polypeptide comprising from the N-terminus:

the type III effector moiety;

a linker peptide;

the translocation domain; and

the binding domain.

In such a construct, the C-terminus of the type III effector protein is fused to the N-terminus of the translocation domain via the linker peptide. An example of such a linker peptide is the sequence CGLVPAGSGP which contains the thrombin protease cleavage site and a cysteine residue for disulphide bridge formation. The latter single chain fusion protein may then be treated with thrombin to give a dichain protein in which the type III effector is linked to the translocation domain by a disulphide link. In another example of a linker peptide in which the translocation domain does not contain a free cysteine residue near its C-terminus, such as is the case when the translocation domain is a fusogenic peptide, the linker peptide contains both cysteine residues required for the disulphide bridge. An example of the latter linker peptide is the amino acid sequence: CGLVPAGSGPSAGSSAC.

In an example of the construct of the invention in which the type III effector moiety is SigD from Salmonella spp produced by recombinant technology, the construct may consist of polypeptide containing (from the N-terminus) the following domains:

the SigD type III effector moiety;

linker peptide (sequence CGLVPAGSGP) to enable attachment of the SigD effector to the translocation domain via a disulphide bridge;

the translocation domain from diphtheria toxin (residues 194-386); and

the binding domain (H_{c} domain) from botulinum type A neurotoxin (residues 872-1296).

The constructs of the invention may also be produced using chemical cross-linking methods. Various strategies are known by which type III effector proteins can be linked to a polypeptide consisting of the translocation domain and binding domain using a variety of established chemical cross-linking techniques. Using these techniques a variety of type III effector constructs can be produced. The type III effector protein is, for example, derivatised with the cross-linking reagent N-succinimidyl 3-[2-pyridyldithio] propionate. The derivatised type III effector is then linked to a peptide containing a translocation domain and binding domain via a free cysteine residue present on the translocation domain.

Protein effectors can be altered to allow their delivery to intracellular compartments other than their usual site of action. For example, mitochondrial or nuclear targeting signals could be added to direct the effector to these compartments. By covalently linking the effector to the targeting domain the effector can be retained in the endosome/lysosome compartment, which would not normally be accessible by bacterial delivery. Effectors can be targeted to specific membrane locations via lipid modifications including myristoylation, palmitoylation, or the addition of short proteins domains that might include SH2, SH3, WW domains, fragments of Rab proteins or synaptojanin-like domains. Those practised in the art would recognise that these targeting strategies offer an advantage for certain therapeutic strategies.

Constructs of the invention may be introduced into either neuronal or non-neuronal tissue using methods known in the art. By subsequent specific binding to neuronal cell tissue, the targeted construct exerts its therapeutic effects. Ideally, the construct is injected near a site requiring therapeutic intervention.

The construct of the invention may be produced as a suspension, emulsion, solution or as a freeze dried powder depending on the application and properties of the therapeutic substance. The construct of the invention may be resuspended or diluted in a variety of pharmaceutically acceptable liquids depending on the application.

“Clostridial neurotoxin” means either tetanus neurotoxin or one of the seven botulinum neurotoxins, the latter being designated as serotypes A, B C₁, D, E, F or G, and reference to the domain of a toxin is intended as a reference to the intact domain or to a fragment or derivative thereof which retains the essential function of the domain.

“Conjugate” means, in relation to two polypeptides, that the polypeptides are linked by a covalent bond, typically forming a single polypeptide as a result, or by a di-sulphide bond.

“Binding domain” means a polypeptide which displays high affinity binding specific to a target cell, e.g. neuronal cell binding corresponding to that of a clostridial neurotoxin. Examples of binding domains derived from clostridial neurotoxins are as follows:

| | |
|---|---|
| Botulinum type A neurotoxin | amino acid residues (872-1296) |
| Botulinum type B neurotoxin | amino acid residues (859-1291) |
| Botulinum type C neurotoxin | amino acid residues (867-1291) |
| Botulinum type D neurotoxin | amino acid residues (863-1276) |
| Botulinum type E neurotoxin | amino acid residues (846-1252) |
| Botulinum type F neurotoxin | amino acid residues (865-1278) |
| Botulinum type G neurotoxin | amino acid residues (864-1297) |
| Tetanus neurotoxin | amino acid residues (880-1315) |

“High affinity binding specific to neuronal cell corresponding to that of a clostridial neurotoxin” refers to the ability of a ligand to bind strongly to cell surface receptors of neuronal cells that are involved in specific binding of a given neurotoxin. The capacity of a given ligand to bind strongly to these cell surface receptors may be assessed using conventional competitive binding assays. In such assays radiolabelled clostridial neurotoxin is contacted with neuronal cells in the presence of various concentrations of non-radiolabelled ligands. The ligand mixture is incubated with the cells, at low temperature (0-3° C.) to prevent ligand internalization, during which competition between the radiolabelled clostridial neurotoxin and non-labelled ligand may occur. In such assays when the unlabelled ligand used is the same as that of the labelled neurotoxin, the radiolabelled clostridial neurotoxin will be displaced from the neuronal cell receptors as the concentration of non-labelled neurotoxin is increased. The competition curve obtained in this case will therefore be representative of the behaviour of a ligand which shows “high affinity binding specificity to neuronal cells corresponding to that of a clostridial neurotoxin”, as used herein.

A carrier that “targets” a particular cell generally does so due to binding of the carrier, or a portion thereof, to that cell and, by way of example, many different ligands with given cell type specificity are described herein.

“Translocation domain” means a domain or fragment of a protein which effects transport of itself and/or other proteins and substances across a membrane or lipid bilayer. The latter membrane may be that of an endosome where translocation will occur during the process of receptor-mediated endocytosis. Translocation domains can frequently be identified by the property of being able to form measurable pores in lipid membranes at low pH (Shone et al. Eur J. Biochem. 167, 175-180). Examples of translocation domains are set out in more detail below:

| | |
|---|---|
| Diphtheria toxin | amino acid residues (194-386) |
| Botulinum type A neurotoxin | amino acid residues (449-871) |
| Botulinum type B neurotoxin | amino acid residues (441-858) |
| Botulinum type C neurotoxin | amino acid residues (442-866) |
| Botulinum type D neurotoxin | amino acid residues (446-862) |
| Botulinum type E neurotoxin | amino acid residues (423-845) |
| Botulinum type F neurotoxin | amino acid residues (440-864) |
| Botulinum type G neurotoxin | amino acid residues (442-863) |
| Tetanus neurotoxin | amino acid residues (458-879) |

Translocation domains are frequently referred to herein as “H_{N} domains”.

“Translocation” in relation to translocation domain, means the internalization events that occur after binding to the cell surface. These events lead to the transport of substances into the cytosol of target cells.

“Injected effector secreted by type III or type IV secretion system” means bacterial proteins that are injected into host cells (mammalian, plant, insect, fish or other) via a modified pilus or “needle-like” injection system frequently referred to as type III or type IV secretion systems” and the term embraces fragments, modifications and variations thereof that retain the essential effector activity.

The invention thus uses modification of intracellular signalling for promoting neuronal growth. Many of the effectors and inhibitors that control the development of the growth cone act through common intracellular signalling pathways that modulate the phosphorylation state of cytoskeletal components and that ultimately determine whether the axon grows or collapses. The appropriate manipulation of intracellular signalling is therefore a powerful approach for eliminating the need for multiple inhibitors of the many factors shown to induce apoptosis and growth cone collapse. The up-regulation of transcription factors that inhibit apoptosis is an example of manipulation of intracellular signalling to promote neural regeneration.

Strategies for therapeutic intervention using the effectors and compositions of the invention include the delivery of agents to eliminate stress-inducing factors and the modification of intracellular signalling to promote cell survival. The latter approach is particularly powerful and the present invention describes conjugates with type III effector moieties which allow such strategies to be pursued.

The constructs of this invention use a specific targeting system to ensure delivery of the therapeutic agent to the desired cells and uses bacterial toxins that have evolved to regulate key stages in the cell signalling machinery of the cells. This strategy offers a number of advantages over other drug platforms. The cell specificity ensures that any alterations in cell signalling occur only in the cells where this modification is desirable and not in other adjacent cells. For example, in neuronal cell-targeted constructs, changes in signalling would only take place in neurones and not in adjacent glial cells where such changes might not be desirable. By targeting key intermediates in the signalling pathway it is possible to co-ordinately regulate a number of overlapping cellular events to promote the desired effect. For example, the activation of Akt by SigD causes an effect on cells by coordinating a number of signalling pathways to actively promote cell survival and block the induction of apoptosis in response to stress factors. This is also a good example of an effector that activates a component of a cell-signalling pathway. Most drug discovery approaches tend to identify inhibitors of specific components.

The invention is now illustrated in the following specific examples.

### EXAMPLES

### Example 1

### Cloning and Expression of Type III Effector Genes

Standard molecular biology protocols were used for all genetic manipulations (Sambrook et al 1989, Molecular cloning; A laboratory manual. Second Edition, Cold Spring Harbor Laboratory Press, New York.). Genes encoding Type III effectors, truncated versions removing the N-terminal hydrophobic domain (e.g removal of amino acids 1-28 for SigD, 1-69 for SptP, 1-76 for SopE), or individual sub-domains (e.g. ExoS GAP domain amino acids 96-234 and ADP-ribosyltransferase domain amino acids 232-453), were amplified from genomic DNA by PCR to generate suitable restriction sites for cloning. In some cases synthetic genes were prepared with codon usage optimised for expression in E.coli. Restriction enzymes such as BamHI (5′) and BglII (3′) were used for cloning with reading frames maintained. Constructs were sequenced to confirm the presence of the correct sequence. Constructs for expression were subcloned, as a suitable fragment, into an expression vector carrying a T7 polymerase promoter site (e.g. pET28, pET30 or derivatives (Novagen Inc, Madison, Wis.)), to generate a fusion with maltose binding protein (e.g. pMALc2x (NEB)) or into other expression vectors known to those familiar with the art. Clones with confirmed sequences were used to transform expression hosts: For T7 polymerase vectors E.coli BL21 (DE3) (Studier and Moffatt 1986 Journal of Molecular Biology 189:113-130) JM109 (DE3) or equivalent strains with a DE3 lysogen. For pMalc2x JM109, BL21, TG1, TB1 or other suitable expression strains.

In addition to the expression of type III effectors as standard fusion proteins an additional approach was used to generate fusion proteins. The type III effector or truncated effector generated as above were cloned into the 5′ end of a gene encoding a cell targeting ligand, which include toxin fragments, antibodies, growth factors, lectins, interleukins, peptides. These fusion proteins were cloned and expressed as either 6-His tagged, MBP tagged or other fusions as described above.

Expression cultures were grown in Terrific Broth containing 30 μg/ml kanamycin and 0.5% (w/v) glucose to an OD₆₀₀ of 2.0 and protein expression was induced with 500 μM IPTG for 2 hours. Cells were lysed by either sonication or suitable detergent treatment (e.g. Bugbuster reagent; Novagen), cell debris pelleted by centrifugation and the supernatant loaded onto a metal chelate column charged with Cu²⁺ (Amersham-Pharmacia Biotech, Uppsala, Sweden).

The recombinant proteins expressed from pET vectors contain amino-terminal histidine (6-His) and T7 peptide tags allowing proteins to be purified by affinity chromatography on either a Cu²⁺ charged metal chelate column. After loading proteins on the column and washing, proteins were eluted using imidazole. All buffers were used as specified by manufacturers. Where appropriate removal of the purification tag was carried out according to manufacturers instructions.

MBP fusions were purified on amylose resin columns as described by the manufacturer (NEB) following growth in Terrific Broth containing 100 μg/ml ampicillin and lysis as described above.

Other fusion systems were used according to manufacturer's instructions and purification carried out on suitable columns using defined methods.

### Example 2

### Production of Recombinant Targeting Vectors Consisting of Translocation and Binding Domains

Standard molecular biology protocols were used for all genetic manipulations (Sambrook et al 1989, Molecular cloning; A laboratory manual. Second Edition, Cold Spring Harbor Laboratory Press, New York.) Clostridial neurotoxin binding domains (BoNT/Hc or TeNT/Hc) derived from either their native genes or synthetic genes with codon usage optimised for expression in E.coli were amplified by PCR. Introduced BamHI (5′) restriction sites and HindIII, SalI or EcoRI (3′) sites were used for most cloning operations with reading frames designed to start with the first base of the restriction site. Constructs were sequenced to confirm the presence of the correct sequence. The translocation domain of diphtheria toxin (DipT) was amplified from its native gene to introduce BamHI and BglII sites at the 5′ and 3′ ends respectively. This BamHI and BglII fragment was subcloned into the BamHI site at the 5′ end of the Hc fragment to generate an in-frame fusion. The entire heavy chain fragment (DipT-Hc) was excised as a BamHI-HindIII or BamHI-SalI or BamHI-EcoRI fragment and subcloned into a suitable expression vector.

Constructs for expression were subcloned into either an expression vector carrying a T7 polymerase promoter site (e.g. pET28, pET30 or derivatives (Novagen Inc, Madison, Wis.)) or to generate a fusion with maltose binding protein (e.g. pMALc2x (NEB)) as a suitable fragment. Clones with confirmed sequences were used to transform expression hosts: For T7 polymerase vectors E.coli BL21 (DE3) (Studier and Moffatt 1986 Journal of Molecular Biology 189:113-130) JM109 (DE3) or equivalent strains with a DE3 lysogen. For pMalc2x JM109, BL21, TG1, TB1 or other suitable expression strains.

The recombinant proteins expressed from pET vectors contain amino-terminal histidine (6-His) and T7 peptide tags allowing proteins to be purified by affinity chromatography on either a Cu²⁺ charged metal chelate column. Expression cultures were grown in Terrific Broth containing 30microg/ml kanamycin and 0.5% (w/v) glucose to an OD₆₀₀ of 2.0 and protein expression was induced with 500 microM IPTG for 2 hours. Cells were lysed by either sonication or suitable detergent treatment (e.g. Bugbuster reagent; Novagen), cell debris pelleted by centrifugation and the supernatant loaded onto a metal chelate column charged with Cu²⁺ (Amersham-Pharmacia Biotech, Uppsala, Sweden). After loading proteins on the column and washing, proteins were eluted using imidazole. All buffers were used as specified by manufacturers. Where appropriate removal of the purification tag was carried out according to manufacturers instructions.

MBP fusions were purified on amylose resin columns as described by the manufacturer (NEB) following growth in Terrific Broth containing 100 microg/ml ampicillin and lysis as described above.

Thrombin or factor Xa protease sites were included within the protein for subsequent removal of these purification tags.

Additional sequences for adding affinity purification tags and one or more specific protease sites for the subsequent removal of these affinity tags may also be included in the reading frame of the gene products.

Other coding sequences that enable expression of the desired protein would also be acceptable. Other tags or linking sites may also be incorporated into the sequence.

Using the techniques described above, targeting vector fragments were constructed by fusing domains of the H_{c} fragments of either botulinum type A, type F or tetanus neurotoxins with the translocation domain of diphtheria toxin.

### Example 3

### Preparation of Botulinum Heavy Chains by Chemical Methods

The various serotypes of the clostridial neurotoxins may be prepared and purified from various toxigenic strains of Clostridium botulinum and Clostridium tetani by methods employing standard protein purification techniques as described previously (Shone and Tranter 1995, Current Topics in Microbiology, 194, 143-160; Springer). Samples of botulinum neurotoxin (1 mg/ml) are dialysed against a buffer containing 50 mM Tris-HCl pH 8.0, 1M NaCl and 2.5M urea for at least 4 hours at 4° C. and then made 100 mM with dithiothreitol and incubated for 16 h at 22° C. The cloudy solution, which contains precipitated light chain, is then centrifuged at 15000×g for 2 minutes and the supernatant fluid containing the heavy chain retained and dialysed against 50 mM HEPES pH 7.5 containing 0.2M NaCl and 5 mM dithiothreitol for at least 4 hours at 4° C. The dialysed heavy chain is centrifuged at 15000×g for 2 minutes and the supernatant retained and dialysed thoroughly against 50 mM HEPES pH 7.5 buffer containing 0.2M NaCl and stored at −70° C. The latter procedure yields heavy chain >95% pure with a free cysteine residue which can be used for chemical coupling purposes. Biological (binding) activity of the heavy chain may be assayed as described in Example 5.

The heavy chains of the botulinum neurotoxins may also be produced by chromatography on QAE Sephadex as described by the methods in Shone and Tranter (1995) (Current Topics in Microbiology, 194, 143-160; Springer).

### Example 4

### Chemical Conjugation of Proteins

Recombinant SigD type III effector from Salmonella spp. was cloned and purified as described in Example 1. The SigD type III effector was chemically modified by treatment with a 3-5 molar excess of N-succinimidyl 3-[2-pyridyidithio] propionate (SPDP) in 0.05M Hepes buffer pH 7.0 containing 0.1M NaCl for 60 min at 22° C. The excess SPDP was removed by dialysis against the same buffer at 4° C. for 16 h. The substituted SigD effector was then mixed in a 1:1 ratio and incubated at 4° C. for 16 h with a targeting vector comprising a translocation domain (with an available free cysteine residue) and a neuronal targeting domain (see Example 2). The latter may also be native heavy chain purified from Clostridium botulinum type A neurotoxin purified as described in Example 3. During the incubation period the SigD effector was conjugated to the targeting vector fragment by a free sulphydryl group. After incubation, the SigD-construct was purified by gel filtration chromatography on Sephadex G200.

### Example 5

### Assay of the Biological Activity of Constructs—Demonstration of High Affinity Binding to Neuronal Cells

Clostridial neurotoxins may be labelled with 125-iodine using chloramine-T and its binding to various cells assessed by standard methods such as described in Evans et al. 1986, Eur J. Biochem., 154, 409 or Wadsworth et al. 1990, Biochem. J. 268, 123). In these experiments the ability of Type III constructs to compete with native clostridial neurotoxins for receptors present on neuronal cells or brain synaptosomes was assessed. All binding experiments were carried out in binding buffers. For the botulinum neurotoxins this buffer consisted of: 50 mM HEPES pH 7.0, 30 mM NaCl, 0.25% sucrose, 0.25% bovine serum albumin. For tetanus toxin, the binding buffer was: 0.05M tris-acetate pH 6.0 containing 0.6% bovine serum albumin. In a typical binding experiment the radiolabelled clostridial neurotoxin was held at a fixed concentration of between 1-20 nM. Reaction mixtures were prepared by mixing the radiolabelled toxin with various concentrations of unlabelled neurotoxin or construct. The reaction mixtures were then added to neuronal cells or rat brain synaptosomes and then incubated at 0-3° C. for 2 hr. After this period the neuronal cells of synaptosomes were washed twice with binding ice-cold binding buffer and the amount of labelled clostridial neurotoxin bound to cells or synaptosomes was assessed by ã-counting. In an experiment using an Type III effector construct what contained the binding domain from botulinum type A neurotoxin, the construct was found to compete with ¹²⁵I-labelled botulinum type A neurotoxin for neuronal cell receptors in a similar manner to unlabelled native botulinum type A neurotoxin. These data showed that the construct had retained binding properties of the native neurotoxin.

### Example 6

### Recombinant Type III Effector Constructs

Recombinant Type III effector-targeting vector constructs were prepared comprising a combination of the following elements:

a type III effector (e.g. SigD from Salmonella spp.)

a linker region, which allows the formation of a disulphide bond between the type III effectors and the translocation domain and which also contains a unique protease cleavage site for cleavage by factor Xa or thrombin to allow the formation of a dichain molecule;

a translocation domain from diphtheria toxin or a endosomolytic (fusogenic) peptide from influenza virus haemagglutinin); and

a neuronal cell-specific binding domain (e.g. from tetanus or botulinum neurotoxin type A or botulinum neurotoxin type F).

The protein sequences of these various domains form specific embodiments of the invention and are shown below the examples.

To confirm the nature of their structure, the recombinant Type III effector-targeting vector constructs were converted to the dichain form by treatment with a unique protease corresponding to the cleavage site sequences within the linker region. Conjugates containing the thrombin cleavage site were treated with thrombin (20 microg per mg of conjugate) for 20 h at 37° C.; conjugates containing the factor Xa cleavage site were treated with factor Xa (20 microg per mg of conjugate) for 20 min at 22° C.

On SDS-PAGE gels, under non-reducing conditions, the majority of Type III effector-targeting vector construct appeared as single band. In the presence of reducing agent (dithiothreitol) two bands were observed corresponding to the type III effector and targeting vector (translocation and binding domains). These data illustrate that, after treatment with the unique protease, the conjugates consist of the latter two components which are linked by a disulphide bridge.

### Example 7

### Formation of Type III Effector Constructs from Type III Effector-Diphtheria Toxin A (CRM197) Fusion Proteins

Type III effector-targeting vector constructs may also formed in vitro by reconstitution from two recombinant fragments. These are:

A Type III effector fused to inactive diphtheria fragment A (CRM197) as described in Example 1.

A recombinant targeting vector in which the translocation domain of diphtheria toxin is fused to a neuronal targeting domain such as that from a clostridial neurotoxin. Production of these is described in Example 2.

Type III effector constructs may be formed by mixing fragments 1 and 2 in equimolar proportions in the presence of 10 mM dithiothreitol and them completely removing the reducing agent by dialysis against phosphate buffered saline at pH 7.4 followed by dialysis against HEPES (0.05M, pH 7.4) containing 0.15 M NaCl. As described above in Example 6, these constructs appear as a single band in SDS gels under non-reducing conditions and two bands in the presence of a reducing agent.

### Example 8

### Formulation of the Type III Effector Construct for Clinical Use

In a formulation of the Type III effector construct for clinical use, recombinant Type III effector construct would be prepared under current Good Manufacturing Procedures. The construct would be transferred, by dilution, to a solution to give the product stability during freeze-drying. Such a formulation may contain Type III effector construct (concentration between 0.1-10 mg/ml) in 5 mM HEPES buffer (pH 7.2), 50 mM NaCl, 1% lactose. The solution, after sterile filtration, would be aliquotted, freeze-dried and stored under nitrogen at −20° C.

### Example 9

### Production of Constructs with Neuroprotective Properties

SigD was cloned (without the first 29 condons) using the methods outlined in Example 1. The protein was expressed and purified either as a fusion with maltose binding protein (e.g. using pMALc2x) or with a Histidine6 (e.g. using pET28a). Purification tags were then removed by standard procedures after affinity purication of the fusion protein. Chemical constructs of SigD were prepared as outlined in Example 4.

A recombinant construct of the invention containing SigD linked to the translocation domain and binding domain of botulinum type A neurotoxin was prepared as outlined in Example 6 using the standard molecular biology procedures outlined in Example 1.

Application of the above constructs to neuronal cells leads to the receptor-mediated internalisation of SigD and subsequent activation of Akt Kinase. Such cells have an enhanced ability to withstand stress such as growth factor removal.

### Example 10

### Constructs for the Treatment of Neurodenerative Disease

Constructs for treatment of neurodegenerative disease and containing the effectors SpiC, SptP or SopE2 were prepared as outlined in Example 9.

### Example 11

### Constructs for Regulating Cellular Secretion and Expression of Cell Surface Receptors

For neuronal cells, constructs containing the effectors SpiC, SopE, RalF, SseE,F,G and J, PipA and B, SifA and B were prepared as outlined in Example 9.

For non-neuronal cells, the targeting domain may be replaced by a ligand with specificity for the target cell type. Such ligands may be selected from a list including: antibodies, carbohydrates, vitamins, hormones, cytokines, lectins, interleukins, peptides, growth factors, cell attachment proteins, toxin fragments, viral coat proteins.

### Example 12

### Constructs for the Treatment of Intracellular Pathogens

Constructs containing the effectors SopE/SopE2, RalF, SpiC, SseE,F,G or J, PipA or B, SifA or B, or other effectors, for example those described in table 1, are useful therapeutic agents for treatment of disease.

Constructs were prepared essentially as described in example 9 but with a suitable binding domain selected from a list including; antibodies, carbohydrates, vitamins, hormones, cytokines, lectins, interleukins, peptides, growth factors, cell attachment proteins, toxin fragments, viral coat proteins etc. For targeting to macrophages this might include protective antigen from Bacillus anthracis or a carbohydrate moiety such as a mannose modification allowing specific uptake.

A recombinant construct of the invention includes an effector protein and a binding domain suitable for targeting the effector to a desired cell type.

When delivered to cells such constructs result in cellular events that cause the death of the intracellular pathogen, prevent its release from the infected cell type or otherwise reduce its ability to infect and cause disease.

Further embodiments of the invention are represented by all combinations of the recited effectors with the recited linker-translocation domain-binding domain conjugates.

The present application includes a sequence listing in which the following sequences referred to by their SEQ ID No.s represent the following embodiments of the invention:

| SEQ ID. | |
|---|---|
| NO. | DESCRIPTION |
| 1 | Diphtheria toxin translocation domain |
| 2 | Diphtheria toxin translocation domain, TeNT-Hc |
| 3 | Thrombin linker, Diphtheria toxin translocation domain, |
| | TeNT-Hc |
| 4 | Factor Xa linker, Diphtheria toxin translocation domain, |
| | TeNT-Hc |
| 5 | Diphtheria toxin translocation domain, BoNT/F-Hc |
| 6 | Thrombin linker, Diphtheria toxin translocation domain, |
| | BoNT/F-Hc |
| 7 | Factor Xa linker, Diphtheria toxin translocation domain, |
| | BoNT/F-Hc |
| 8 | AAC46234 invasion gene D protein [Salmonella |
| | typhimurium] SigD |
| 9 | AAF21057 invasion protein D [Salmonella typhimurium] |
| | SopB |
| 10 | CAC05808 IpgD, secreted by the Mxi-Spa machinery, |
| | modulates entry of bacteria into epithelial cells [Shigella |
| | flexneri] |
| 11 | AAC 69766 targeted effector protein [Yersinia pestis] |
| | YopJ |
| 12 | AAC02071 SopE [Salmonella typhimurium] |
| 13 | AAC44349 protein tyrosine phosphatase SptP |
| | [Salmonella typhimurium] |
| 14 | NP_047628 targeted effector [Yersinia pestis] YopE |
| 15 | AAK39624 exoenzyme S [Pseudomonas aeruginosa] |
| 16 | AAG03434 exoenzyme T [Pseudomonas aeruginosa] |
| 17 | NP_047619 Yop targeted effector [Yersinia pestis] YopT |
| 18 | NP_052380 protein kinase YopO [Yersinia enterocolitica] |
| 19 | AAF82095 outer protein AvrA [Salmonella enterica subsp. |
| | enterica serovar Dublin] |
| 20 | AAC44300 SpiC [Salmonella typhimurium] |
| 21 | SigD with the first 29 codons removed, thrombin linker, |
| | diphtheria translocation domain, TeNT-Hc |
| 22 | SigD with the first 29 codons removed, factor Xa linker, |
| | diphtheria translocation domain, TeNT-H_{c} |
| 23 | SigD with the first 29 codons removed, thrombin linker, |
| | diphtheria toxin translocation domain, with BoNT/F-H_{c} |
| 24 | SigD, factor Xa linker, diphtheria toxin translocation |
| | domain, with BoNT/F-H_{c} |
| 25 | YopT, factor Xa linker, diphtheria translocation domain, |
| | TeNT-H_{c} |
| 26 | YopT, factor Xa linker, diphtheria toxin translocation |
| | domain, with BoNT/F-H_{c} |
| 27 | SpiC, thrombin linker, diphtheria translocation domain, |
| | TeNT-Hc |
| 28 | SpiC, factor Xa linker, diphtheria translocation domain, |
| | TeNT-H_{c} |
| 29 | SpiC fused to a domain consisting the N-terminal 254 |
| | residues from Bacillus anthracis lethal factor capable of |
| | interacting with protective antigen |
| 30 | Bacillus anthracis protective antigen |
| 31 | Clostridium botulinum C2 toxin component 1 |
| 32 | Clostridium botulinum C2 toxin component 2 |

TABLE 1
Examples of type III and type IV effectors and their activity.

| Effector | Biochemical function | Possible applications |
|---|---|---|
| YopT Yersinia spp | Inactivates Rho GTPases by | Stimulate nerve regrowth |
| | direct | following damage |
| ExoS (N-terminal domain) | GTPase activating protein for | Stimulate nerve regrowth |
| Pseudomonas aeuruginosa | Rho GTPases | |
| YopE Yersinia spp | | |
| ExoS (C-terminal domain) | ADP-ribosyltranferase | Block Ras/Rap signalling |
| P. aeuruginosa | modifies Ras and Rap | pathways |
| | GTPases | |
| SptP (N-terminal domain) | GAP activity for Rac 1/Cdc | |
| Salmonella spp | 42 | |
| SopE/E2 S. typhimurium | Guanine nucleotide exchange | Regulates nitric oxide |
| | factor for Cdc42/Rac | release |
| YpkO/YopO Yersinia spp | Serine/threonine kinase | |
| | modifies RhoA/Rac | |
| YopP/YopJ Yersinia spp | Blocks activation of various | Induction of apoptosis in |
| AvrXv/AvrBsT Xanthomonas | MAP kinase pathways | tumour cells |
| campestris | | Block release of |
| | | inflammatory mediators |
| | | from damaged cells |
| SopB/SigA/SigD Salmonella | Activate AKT kinase | Block apoptosis in |
| spp | | damaged/ageing |
| IpgD Shigella flexneri | | neurons |
| SpiC S. enterica | Block endosome fusion | Prevent neurotransmitter |
| | | release from pain fibres |
| IpaB | Induces apoptosis by direct | Induction of apoptosis in |
| SipB | activation of caspase 1 | glioma/neuroblastoma |
| | | cells |
| Orf19 E. coli | Affects mitochondrial function | Modulation of induction |
| IpgB Shigella flexneri | | of cell death and other |
| | | mitochondrial functions |
| Unidentified effector | Blocks apoptosis | Prevent apoptosis in |
| Chlamydia spp | | damaged/ageing |
| | | neurones |
| RalF Listeria monocytogenes | Guanine nucleotide exchange | Promote or prevent |
| | factor for ARF | membrane |
| | | compartment fusion |
| SpiC, SopE, SseE, F, G or J, | Various | Treating intracellular |
| PipA or B, SifA or B, | | pathogens or disorders |
| Salmonella spp. RalF, | | of intracellular |
| Listeria monocytogenes | | trafficking |
| CagA Helicobacter pylori | Cytoskeletal modification | Alter uptake or release |
| | | of membrane vesicle |
| | | contents |
| YopM Yersinia spp, PopC | Leucine rich repeat protein. | Upregulation of genes |
| Ralstonia solanacearum | Possible transcription factors | involved in cell cycle |
| | | and cell growth (YopM) |
| | | or other genes. |

1-48. (canceled)

49. A conjugate comprising an injected bacterial effector protein and a carrier that targets the effector protein to a target cell, wherein the carrier comprises a first domain that targets the effector to a target cell and undergoes receptor-mediated endocytosis, and a second domain that translocates the effector across an endosomal membrane and into the cytosol of the cell.

50. The conjugate of claim 49, wherein said effector protein is linked by a linker to the carrier.

51. The conjugate of claim 50, wherein said linker is cleavable, in that it can be cleaved after entry into the target cell so as to release the effector from the carrier.

52. The conjugate of claim 49, wherein said carrier targets the effector to a cell selected from a group consisting of an epithelial cell, a neuronal cell, a secretory cell, an immunological cell, an endocrine cell, an inflammatory cell, an exocrine cell, a bone cell and a cell of the cardiovascular system.

53. The conjugate of claim 49, wherein said conjugate is a single polypeptide.

54. The conjugate of claim 49, wherein said first domain is selected from (a) neuronal cell binding domains of clostridial toxins; and (b) fragments, variants and derivatives of the domains in (a) that substantially retain the neuronal cell binding activity of the domains of (a).

55. The conjugate of claim 49, wherein said second domain is selected from (a) domains of clostridial neurotoxins that translocate polypeptide sequences into cells, and (b) fragments, variants and derivatives of the domains of (a) that substantially retain the translocating activity of the domains of (a).

56. The conjugate of claim 49, wherein said second domain is selected from:
(a) a translocation domain that is not a H_{N} domain of a clostridial toxin and is not a fragment or derivative of a H_{N} domain of a clostridial toxin;
(b) a non-aggregating translocation domain as measured by size in physiological buffers;
(c) a H_{N} domain of a diphtheria toxin,
(d) a fragment or derivative of (c) that substantially retains the translocating activity of the H_{N} domain of a diphtheria toxin,
(e) a fusogenic peptide,
(f) a membrane disrupting peptide, and
(g) translocating fragments and derivatives of (e) and (f).

57. The conjugate of claim 49, wherein said linker is cleaved in the neuronal cell so as to release the effector protein from the targeting component.

58. The conjugate of claim 49, wherein said linker is a disulphide bridge or a site for a protease found in the target cell.

59. The conjugate of claim 49, wherein said injected bacterial effector protein has an activity selected from a group consisting of activating GTPase, inactivating GTPase, enhancing replacement of bound GDP by GTP, causing covalent modification of GTPase, protein kinase activity, protein phosphatase, inositol phosphatase activity, inhibition of mitogen activated protein kinase kinase, regulation of gene expression, transcription factor and modulation of cellular trafficking.

60. A pharmaceutical composition comprising the conjugate of claim 49.

61. The pharmaceutical composition of claim 60, for a treatment selected from the group consisting of promoting survival of cells, preventing damage to cells, reversing damage to cells, promoting growth of cells, inhibiting apoptosis, inhibiting release of an inflammatory mediator from cells, promoting division of cells and treating intracellular infection.

62. The pharmaceutical composition of claim 61, for treating neuronal cells.

63. The pharmaceutical composition of claim 61, wherein said composition promotes the survival of neuronal cells.

64. The pharmaceutical composition of claim 55, wherein said activity is treating intracellular infection.

65. The pharmaceutical composition of claim 60, for a treatment selected from the group consisting of inhibiting survival of cells, inhibiting growth of cells, inhibiting division of cells, promoting apoptosis, killing cells, promoting release of an inflammatory mediator from cells, regulating nitric oxide release from cells, inhibiting secretion from cells, interfering with intracellular trafficking and modulating expression of cell-surface markers.

66. The pharmaceutical composition of claim 65, wherein said activity is interfering with intracellular trafficking.

67. The pharmaceutical composition of claim 65, wherein said activity is modulating expression of cell-surface markers.

68. The pharmaceutical composition of claim 65, wherein said activity is inhibiting secretion from cells.

69. A method of treating a mammal in need thereof, comprising administering to said mammal the pharmaceutical composition of claim 60.

70. A method of preparing the conjugate of claim 49, comprising combining said effector protein with the carrier.

71. The method of claim 70, comprising chemically linking the effector protein with the carrier.

72. A method of preparing the conjugate of claim 49, comprising expressing a DNA that encodes a polypeptide having a first region that corresponds to the effector protein and a second region that codes for the carrier.

73. The method of claim 72, wherein said carrier comprises a third region, between the first and second regions, which is cleaved by a proteolytic enzyme present in the target cell.

74. The method of claim 70, comprising linking said carrier between the first and second region and linking the first and second regions via a disulphide bridge.

75. A DNA construct encoding the conjugate of claim 49.

76. A pharmaceutical composition, comprising the DNA construct of claim 75.

77. A pharmaceutical composition comprising a vector containing the DNA construct of claim 75.

78. A method of treating a mammal in need thereof, comprising administering to said mammal the pharmaceutical composition of claim 76.

79. A pharmaceutical composition comprising an injected bacterial effector protein.

80. A method of treating intracellular infection comprising administering to a mammal the pharmaceutical composition of claim 79.

81. A method of interfering with intracellular trafficking comprising administering to a mammal the pharmaceutical composition of claim 79.

82. A method of modulating expression of cell-surface markers comprising administering to a mammal the pharmaceutical composition of claim 79.

83. A method of inhibiting secretion from cells comprising administering to a mammal the pharmaceutical composition of claim 79.

84. A method of treating a neuronal cell comprising administering to a mammal the pharmaceutical composition of claim 79.


SEQUENCE LISTING (32 sequences)

SEQ ID NO: 1 - protein, 210 aa, Corynebacterium diphtheria
SVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRS

SEQ ID NO: 2 - protein, 665 aa, Artificial sequence
  diphtheria toxin translocation domain TeNT-HC
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 3 - protein, 677 aa, Artificial sequence
  thrombin linker, diphtheria toxin translocation domain, TeNT-HC
RSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 4 - protein, 677 aa, Artificial sequence
  factor Xa linker, Diphtheria toxin translocation domain, TeNT-HC
RSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 5 - protein, 645 aa, Artificial sequence
  diphtheria toxin translocation domain with BoNT/F-HC
GSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 6 - protein, 657 aa, Artificial sequence
  thrombin linker, diphtheria toxin translocation domain, BoNT/F-HC
RSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 657 aa, Artificial sequence
  factor Xa linker, diphtheria toxin translocation domain, BoNT/F-Hc
RSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 8 - protein, 563 aa, Salmonella typhimurium
MQIQSFYHSASLKTQEAFKSLQKTLYNGMQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFFDGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKGEIISLHQTHMLSAPGSLPDSGGQKIFQKVLLNSGNLEIQKQNTGGAGNKVMKNLSPEVLNLSYQKRVGDENIWQSVKGISSLITS

SEQ ID NO: 9 - protein, 433 aa, Salmonella typhimurium
VLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFCGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKREIISLHQTHMLSAPGSLPDSGGQKIFQKVLLNSGNLEIQKQNTGGAGNKVMKN

SEQ ID NO: 10 - protein, 538 aa, Shigella flexneri
MHITNLGLHQVSFQSGDSYKGAEETGKHKGVSVISYQRVKNGERNKGIEALNRLYLQNQTSLTGKSLLFARDKAEVFCEAIKLAGGDTSKIKAMMERLDTYKLGEVNKRHINELNKVISEEIRAQLGIKNKKELQTKIKQIFTDYLNNKNWGPVNKNISHHGKNYSFQLTPASHMKIGNKNIFVKEYNGKGICCASTRERDHIANMWLSKVVDDEGKEIFSGIRHGVISAYGLKKNSSERAVAARNKAEELVSAALYSRPELLSQALSGKTVDLKIVSTSLLTPTSLTGGEESMLKDQVSALKGLNSKRGGPTKLLIRNSDGLLKEVSVNLKVVTFNFGVNELALKMGLGWRNVDKLNDESICSLLGDNFLKNGVIGGWAAEAIEKNPPCKNDVIYLANQIKEIVNNKLQKNDNGEPYKLSQRVTLLAYTIGAVPCWNCKSGKDRTGMQDAEIKREIIRKHETGQFSQLNSKLSSEEKRLFSTILMNSGNMEIQEMNTGVPGNKVMKKLPLSSLELSYSERIGDPKIWNMVKGYSSFV

SEQ ID NO: 11 - protein, 288 aa, Yersinia pestis
MIGPISQINISGGLSEKETSSLISNEELKNIITQLETDISDGSWFHKNYSRMDVEVMPALVIQANNKYPEMNLNLVTSPLDLSIEIKNVIENGVRSSRFIINMGEGGIHFSVIDYKHINGKTSLILFEPANFNSMGPAMLAIRTKTAIERYQLPDCHFSMVEMDIQRSSSECGIFSLALAKKLYIERDSLLKIHEDNIKGILSDGENPLPHDKLDPYLPVTFYKHTQGKKRLNEYLNTNPQGVGTVVNKKNETIVNRFDNNKSIVDGKELSVSVHKKRIAEYKTLLKV

SEQ ID NO: 12 - protein, 180 aa, Salmonella typhimurium
MTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAYASQTREAILSAVYSKNKDQCCNLLISKGINIAPFLQEIGEAAKNAGLPGTTKNDVFTPSGAGANPFITPLISSA

SEQ ID NO: 13 - protein, 543 aa, Salmonella typhimurium
MLKYEERKLNNLTLSSFSKVGVSNDARLYIAKENTDKAYVAPEKFSSKVLTWLGKMPLFKNTEVVQKHTENIRVQDQKILQTFLHALTEKYGETAVNDALLMSRINMNKPLTQRLAVQITECVKAADEGFINLIKSKDNVGVRNAALVIKGGDTKVAEKNNDVGAESKQPLLDIALKGLKRTLPQLEQMDGNSLRENFQEMASGNGPLRSLMTNLQNLNKIPEAKQLNDYVTTLTNIQVGVARFSQWGTCGGEVERWVDKASTHELTQAVKKIHVIAKELKNVTAELEKIEAGAPMPQTMSGPTLGLARFAVSSIPINQQTQVKLSDGMPVPVNTLTFDGKPVALAGSYPKNTPDALEAHMKMLLEKECSCLVVLTSEDQMQAKQLPPYFRGSYTFGEVHTNSQKVSSASQGEAIDQYNMQLSCGEKRYTIPVLHVKNWPDHQPLPSTDQLEYLADRVKNSNQNGAPGRSSSDKHLPMIHCLGGVGRTGTMAAALVLKDNPHSNLEQVRADFRDSRNNRMLEDASQFVQLKAMQAQLLMTTAS

SEQ ID NO: 14 - protein, 219 aa, Yersinia pestis
MKISSFISTSLPLPTSVSGSSSVGEMSGRSVSQQTSDQYANNLAGRTESPQGSSLASRIIERLSSVAHSVIGFIQRMFSEGSHKPVVTPAPTPAQMPSPTSFSDSIKQLAAETLPKYMQQLNSLDAEMLQKNHDQFATGSGPLRGSITQCQGLMQFCGGELQAEASAILNTPVCGIPFSQWGTIGGAASAYVASGVDLTQAANEIKGLAQQMQKLLSLM

SEQ ID NO: 15 - protein, 453 aa, Pseudomonas aeruginosa
MHIQSLQQSPSFAVELHQAASGRLGQIEARQVATPSEAQQLAQRQDAPKGEGLLARLGAALVRPFVAIMDWLGKLLGSHARTGPQPSQDAQPAVMSSAVVFKQMVLQQALPMTLKGLDKASELATLTPEGLAREHSRLASGDGALRSLSTALAGIRAGSQVEESRIQAGRLLERSIGGIALQQWGTTGGAASQLVLDASPELRREITDQLHQVMSEVALLRQAVESEVSRVSADKALADGLVKRFGADAEKYLGRQPGGIHSDAEVMALGLYTGIHYADLNRALRQGQELDAGQKLIDQGMSAAFEKSGQAEQVVKTFRGTRGGDAFNAVEEGKVGHDDGYLSTSLNPGVARSFGQGTISTVFGRSGIDVSGISNYKNEKEILYNKETDMRVLLSASDEQGVTRRVLEEAALGEQSGHSQGLLDALDLASKPERSGEVQEQDVRLRMRGLDLA

SEQ ID NO: 16 - protein, 457 aa, Pseudomonas aeruginosa
MHIQSSQQNPSFVAELSQAVAGRLGQVEARQVATPREAQQLAQRQEAPKGEGLLSRLGAALARPFVAIIEWLGKLLGSRAHAATQAPLSRQDAPPAASLSAAEIKQMMLQKALPLTLGGLGKASELATLTAERLAKDHTRLASGDGALRSLATALVGIRDGSRIEASRTQAARLLEQSVGGIALQQWGTAGGAASQHVLSASPEQLREIAVQLHAVMDKVALLRHAVESEVKGEPVDKALADGLVEHFGLEAEQYLGEHPDGPYSDAEVMALGLYTNGEYQHLNRSLRQGRELDAGQALIDRGMSAAFEKSGPAEQVVKTFRGTQGRDAFEAVKEGQVGHDAGYLSTSRDPGVARSFAGQGTITTLFGRSGIDVSEISIEGDEQEILYDKGTDMRVLLSAKDGQGVTRRVLEEATLGERSGHGEGLLDALDLATGTDRSGKPQEQDLRLRMRGLDLA

SEQ ID NO: 17 - protein, 322 aa, Yersinia pestis
MNSIHGHYHIQLSNYSAGENLQSATLTEGVIGAHRVKVETALSHSNLQKKLSATIKHNQSGRSMLDRKLTSDGKANQRSSFTFSMIMYRMIHFVLSTRVPAVRESVANYGGNINFKFAQTKGAFLHKIIKHSDTASGVCEALCAHWIRSHAQGQSLFDQLYVGGRKGKFQIDTLYSIKQLQIDGCKADVDQDEVTLDWFKKNGISERMIERHCLLRPVDVTGTTESEGLDQLLNAILDTHGIGYGYKKIHLSGQMSAHAIAAYVNEKSGVTFFDPNFGEFHFSDKEKFRKWFTNSFWGNSMYHYPLGVGQRFRVLTFDSKEV

SEQ ID NO: 18 - protein, 729 aa, Yersinia enterocolitica
MKIMGTMPPSISLAKAHERISQHWQNPVGELNIGGKRYRIIDNQVLRLNPHSGFSLFREGVGKIFSGKMFNFSIARNLTETLHAAQKTTSQELRSDIPNALSNLFGAKPQTELPLGWKGKPLSGAPDLEGMRVAETDKFAEGESHISIIETKDNQRLVAKIERSIAEGHLFAELEAYKHIYKTAGKHPNLANVHGMAVVPYGNRKEEALLMDEVDGWRCSDTLRSLADSWKQGKINSEAYWGTIKFIAHRLLDVTNHLAKAGIVHNDIKPGNVVFDRASGEPVVIDLGLHSRSGEQPKGFTESFKAPELGVGNLGASEKSDVFLVVSTLLHGIEGFEKDPEIKPNQGLRFITSEPAHVMDENGYPIHRPGIAGVETAYTRFITDILGVSADSRPDSNEARLHEFLSDGTIDEESAKQILKDTLTGEMSPLSTDVRRITPKKLRELSDLLRTHLSSAATKQLDMGVVLSDLDTMLVTLDKAEREGGVDKDQLKSFNSLILKTYSVIEDYVKGREGDTKSSSAEVSPYHRSNFMLSIAEPSLQRIQKHLDQTHSFSDIGSLVRAHKHLETLLEVLVTLSPQGQPVSSETYSFLNRLAEAKVTLSQQLDTLQQQQESAKAQLSILINRSGSWADVARQSLQRFDSTRPVVKFGTEQYTAIHRQMMAAHAAITLQEVSEFTDDMRNFTADSIPLLIRLGRSSLIDEHLVEQREKLRELTTIAERLNRLEREWM

SEQ ID NO: 19 - protein, 129 aa, Salmonella enterica
VMDGKTSVILFEPAACSAFGPALLALRTKAALEREQLPDCYFAMVELDIQRSSSECGIFSLALAKKLQLEFMNLVKIHEDNICERLCGEEPFLPSDKADRYLPVSFYKHTQGVQRLNEYVEANPAAGSS

SEQ ID NO: 20 - protein, 133 aa, Salmonella typhimurium
MSEEGFMLAVLKGIPLIQDIRAEGNSRSWIMTIDGHPARGEIFSEAFSISLFLNDLESLPKPCLAYVTLLLAAHPDVHDYAIQLTADGGWLNGYYTTSSSSELIAIEIEKHLALTCILKNVIRNHHKLYSGGV

SEQ ID NO: 21 - protein, 1212 aa, Artificial sequence
  Protein sequence for SigD with the first 29 codons removed, thrombin linker, diphtheria translocation domain, TeNT-HC
MQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFFDGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKGEIISLHQTHMLSAPGSLPDSGGQKIFQKVLLNSGNLEIQKQNTGGAGNKVMKNLSPEVLNLSYQKRVGDENIWQSVKGISSLITSRSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 22 - protein, 1212 aa, Artificial sequence
  Protein sequence for SigD with the first 29 codons removed, factor Xa linker, diphtheria translocation domain, TeNT-HC
MQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFFDGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKGEIISLHQTHMLSAPGSLPDSGGQKIFQKVLLNSGNLEIQKQNTGGAGNKVMKNLSPEVLNLSYQKRVGDENIWQSVKGISSLITSRSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 23 - protein, 1192 aa, Artificial sequence
  Protein sequence for SigD with the first 29 codons removed, thrombin linker, diphtheria toxin translocation domain, with BoNT/F-HC
MQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFFDGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKGEIISLHQTHMLSAPGSLPDSGGQKIFQKVLLNSGNLEIQKQNTGGAGNKVMKNLSPEVLNLSYQKRVGDENIWQSVKGISSLITSRSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 24 - protein, 1192 aa, Artificial sequence
  Protein sequence for SigD, factor Xa linker, diphtheria toxin translocation domain, with BoNT/F-HC
MQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFFDGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKGEIISLHQTHMLSAPGSLPDSGGQKIFQKVLLNSGNLEIQKQNTGGAGNKVMKNLSPEVLNLSYQKRVGDENIWQSVKGISSLITSRSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 25 - protein, 999 aa, Artificial sequence
  Protein sequence for YopT, factor Xa linker, diphtheria translocation domain, TeNT- HC
MNSIHGHYHIQLSNYSAGENLQSATLTEGVIGAHRVKVETALSHSNLQKKLSATIKHNQSGRSMLDRKLTSDGKANQRSSFTFSMIMYRMIHFVLSTRVPAVRESVANYGGNINFKFAQTKGAFLHKIIKHSDTASGVCEALCAHWIRSHAQGQSLFDQLYVGGRKGKFQIDTLYSIKQLQIDGCKADVDQDEVTLDWFKKNGISERMIERHCLLRPVDVTGTTESEGLDQLLNAILDTHGIGYGYKKIHLSGQMSAHAIAAYVNEKSGVTFFDPNFGEFHFSDKEKFRKWFTNSFWGNSMYHYPLGVGQRFRVLTFDSKEVRSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 26 - protein, 979 aa, Artificial sequence
  Protein sequence for YopT, factor Xa linker, diphtheria toxin translocation domain, with BoNT/F-HC
MNSIHGHYHIQLSNYSAGENLQSATLTEGVIGAHRVKVETALSHSNLQKKLSATIKHNQSGRSMLDRKLTSDGKANQRSSFTFSMIMYRMIHFVLSTRVPAVRESVANYGGNINFKFAQTKGAFLHKIIKHSDTASGVCEALCAHWIRSHAQGQSLFDQLYVGGRKGKFQIDTLYSIKQLQIDGCKADVDQDEVTLDWFKKNGISERMIERHCLLRPVDVTGTTESEGLDQLLNAILDTHGIGYGYKKIHLSGQMSAHAIAAYVNEKSGVTFFDPNFGEFHFSDKEKFRKWFTNSFWGNSMYHYPLGVGQRFRVLTFDSKEVRSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSTMSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 27 - protein, 810 aa, Artificial sequence
  Protein sequence for SpiC, thrombin linker, diphtheria translocation domain, TeNT-HC
MSEEGFMLAVLKGIPLIQDIRAEGNSRSWIMTIDGHPARGEIFSEAFSISLFLNDLESLPKPCLAYVTLLLAAHPDVHDYAIQLTADGGWLNGYYTTSSSSELIAIEIEKHLALTCILKNVIRNHHKLYSGGVRSCGLVPRGSGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 28 - protein, 810 aa, Artificial sequence
  Protein sequence for SpiC, factor Xa linker, diphtheria translocation domain, TeNT- HC
MSEEGFMLAVLKGIPLIQDIRAEGNSRSWIMTIDGHPARGEIFSEAFSISLFLNDLESLPKPCLAYVTLLLAAHPDVHDYAIQLTADGGWLNGYYTTSSSSELIAIEIEKHLALTCILKNVIRNHHKLYSGGVRSCGIEGRAPGPGSSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRSAYSPGHKTQPFLHDGYAVSWNTVRSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDLQ

SEQ ID NO: 29 - protein, 393 aa, Artificial sequence
  Protein sequence for SpiC fused to a domain consisting the N-terminal 254 residues from Bacillus anthracis lethal factor capable of interacting with protective antigen
MSEEGFMLAVLKGIPLIQDIRAEGNSRSWIMTIDGHPARGEIFSEAFSISLFLNDLESLPKPCLAYVTLLLAAHPDVHDYAIQLTADGGWLNGYYTTSSSSELIAIEIEKHLALTCILKNVIRNHHKLYSGGVMNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEP

SEQ ID NO: 30 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 31 - protein, 431 aa, Clostridium botulinum
MPIIKEPIDFINKPESEAQKWGKEEEKRWFTKLNNLEEVAVNQLKTKEDKTKIDNFSTDILFSSLTAIEIMKEDENQNLFDVERIREALLKNTLDREVIGYVNFTPKELGINFSIRDVELNRDISDEILDKVRQQIINQEYTKFSFVSLGLNDNSIDESIPVIVKTRVPTTFNYGVLNNKETVSLLLNQGFSIIPESAIITTIKGKDYILIEGSLSQELDFYNKGSEAWGEKNYGDYVSKLSQEQLGALEGYLHSDYKAINSYLRNNRVPNNDELNKKIELISSALSVKPIPETLIAYRRVDGIPFDLPSDFSFDKKENGEIIADKTKLNEFIDKWTGKEIENLSFSSTSLKSTPLSFSKSRFIFRLRLSEGTIGAFIYGFSGFQDEQEILLNKNSTFKIFRITPITSIINRVTKMTQVVIDAEVIQNKEI

SEQ ID NO: 32 - protein, 721 aa, Clostridium botulinum
MLVSKFENSVKNSNKNYFTINGLMGYYFENDFFNLNIISPTLDGNLTFSKEDINSILGNKIIKSARWIGLIKPSITGEYILSTNSPNCRVELNGEIFNLSLNTSNTVNLIQGNVYDIRIEQLMSENQLLKNYEGIKLYWETSDIIKEIIPSEVLLKPNYSNTNEKSKFIPNNTLFSNAKLKANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTGESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSFSGQTTQVAVVAPNFSDPEDKTPKLTLEQALVKAFALEKKNGKFYFHGLEISKNEKIQVFLDSNTNNDFENQLKNTADKDIMHCIIKRNMNILVKVITFKENISSINIINDTNFGVQSMTGLSNRSKGQDGIYRAATTAFSFKSKELKYPEGRYRMRFVIQSYEPFTCNFKLFNNLIYSSSFDKGYYDEFFYFYYNGSKSFFNISCDIINSINRLSGVFLIELDKLII